(19) United States
(12) Patent Application Publication
(10) Pub. No.: US 2022/0192772 A1
(43) Pub. Date: Jun. 23, 2022

(54) Manipulation Device

(71) Applicant: KARL STORZ SE & Co KG, Tuttlingen (DE)

(72) Inventors: Martin Blocher, Stockach (DE); Sven Axel Grüner, Rietheim (DE); Judith Holzer, Epfendorf (DE); Daniel Kärcher, Radolfzell (DE); Dominik Längle, Mühlheim-Stetten (DE); Robin Merz, Furtwangen (DE); Janosz Schneider, Donaueschingen (DE); Sven Schneider, Tuttlingen (DE); Jochen Stefan, Wald (DE); Tobias Unger, Wurmlingen (DE)

(73) Assignee: KARL STORZ SE & Co KG, Tuttlingen (DE)

(21) Appl. No.: 17/555,878

(22) Filed: Dec. 20, 2021

(30) Foreign Application Priority Data
Dec. 22, 2020 (DE) 102020134601.8

(51) Int. Cl. A61B 34/00; A61B 90/70

(52) U.S. Cl. CPC A61B 34/72; A61B 90/70

(57) ABSTRACT

A manipulation device (14) for a microinvasive medical instrument (10) comprises a first grip member (22) and a second grip member (40), that are movable relative to each other, an irrigation port (90) for receiving an irrigation liquid for cleaning the manipulation device (14) between two uses, and an irrigation channel (92, 94, 96, 98) that connects the irrigation port (90) to one or more outlets (93, 95, 33, 65). The irrigation channel (92, 94, 96, 98) is arranged in the first grip member (22).

### CROSS REFERENCE TO RELATED APPLICATIONS

This application claims priority to German Patent Application No. 102020134601.8, filed Dec. 22, 2020, and entitled, “Handhabungseinrichtung,” and is incorporated herein by reference.

### TECHNICAL FIELD OF THE INVENTION

The present invention relates to a manipulation device for a microinvasive medical instrument and to a microinvasive medical instrument having such a manipulation device.

### BACKGROUND OF THE INVENTION

Many microsurgical instruments are lockable. This is true in particular of microsurgical instruments with gripping tools at their distal ends. The locking is affected almost exclusively at the manipulation device at the proximal end, specifically by force-fit or, more often, form-fit locking. For example, a locking spur with a plurality or multiplicity of locking grooves or locking teeth is provided on one of two grip members that are movable relative to each other, while the other grip member is provided with a latching pawl which can engage in any one of the locking grooves or can engage behind any one of the locking teeth.

To release the lock, a release device is generally provided, for example a release lever, which is directly manually actuatable, specifically movable, in order to move the latching pawl from its locking position, in which it is connected with form-fit and/or force-fit engagement to the locking spur, to a release position. In addition to this, a deactivation device can be provided in order to keep the locking device permanently disengaged. The entire mechanism for the locking action has a complex structure and a complex geometry. A complete clean, which has to take place prior to the sterilization in the case of a reusable instrument, can be correspondingly complex.

It is an object of the present invention to make available an improved manipulation device for a medical instrument.

### SUMMARY OF THE INVENTION

A manipulation device for a medical instrument comprises a first grip member and a second grip member, which are movable relative to each other, an irrigation port for receiving an irrigation liquid for cleaning the manipulation device between two uses, and an irrigation channel which connects the irrigation port to one or more outlets, wherein the irrigation channel is arranged in the first grip member.

The manipulation device is provided and designed in particular for a microinvasive medical instrument, i.e., for an instrument which is in turn provided and designed for use in microinvasive medical procedures. The manipulation device is in particular reusable, i.e., can be repeatedly cleaned and sterilized, in particular autoclaved.

The manipulation device can be an integral component part of a medical instrument. This means that the manipulation device is connected to one or more further component parts of the instrument permanently and in such a way that separation is not possible without using tools or is in any case not reversible, i.e., separation is not possible without destruction.

Alternatively, the manipulation device can be a component part of a medical instrument but can be easily separated from other component parts of the microinvasive medical instrument, in particular without tools and without destruction, that is to say reversibly, and can be connected to them again.

The irrigation port is in particular a Luer connector, i.e., a concave Luer cone according to ISO 80369 or DIN EN 80369-7. In this case, it has a maximum length of 7.5 mm, a taper of 6 percent, that is to say an angle of 3.44 degrees, and a minimum diameter of 4 mm. Irrigation liquid can be conveyed into the irrigation channel through the irrigation port at a pressure of approximately 2 bar, for example.

The irrigation channel is in particular branched, with a plurality of portions each leading to one or more outlets through which the irrigation liquid can emerge from the first grip member. The irrigation channel can have frequently alternating cross sections. Through lateral openings of the cross sections, irrigation liquid can pass from the irrigation channel into adjoining regions of the first grip member.

The delivery of irrigation liquid to an irrigation channel, through which the irrigation liquid can be conveyed into the interior of the first grip member, can facilitate particularly effective cleaning in the interior of the first grip member and especially also at the locations where the irrigation liquid emerges again from the first grip member. A stream of irrigation liquid directed from the inside outwards can particularly effectively remove contamination from the first grip member.

In a manipulation device as described here, the irrigation port is arranged in particular at a side of the first grip member facing towards the second grip member.

During the use of the manipulation device, those sides or surface regions of the grip members facing towards each other are often less contaminated than the sides facing away from each other, i.e., the outwardly oriented surface regions of the grip members. Since the irrigation port itself constitutes an opening in which and through which contamination is possible, the arrangement of the irrigation port at the side of the first grip member facing towards the second grip member can reduce the contamination of the first grip member through the irrigation port.

In a manipulation device as described here, the irrigation port is in particular arranged proximally with respect to a connection region of the grip members.

The connection region of the grip members is in particular the region in which the grip members are mechanically connected to each other by a hinge. The arrangement of the irrigation port proximally with respect to the connection region signifies that, as regards the path along which forces and moments are transmitted from the grip member to a distal region, for example to a shaft and a tool at the distal end of the microinvasive medical instrument, the connection region is arranged between the irrigation port and the shaft. The irrigation port is thus arranged in the region in which a locking device for locking the grip members relative to each other is also typically provided.

In a manipulation device as described here, the irrigation channel is in particular forked and fluidically connects the irrigation port to a plurality of outlets.

Thus, after one or more branches, the irrigation channel can have several portions which are connected in parallel with respect to the stream of fluid and through which an irrigation liquid can reach different regions of the first grip member.

In a manipulation device as described here, the second grip member is movable relative to the first grip member in particular in a plane, the irrigation channel running substantially parallel to the plane.

The grip members are pivotable relative to each other in particular about a pivot axis defined by a hinge between the grip members. The plane in which the second grip member is movable relative to the first grip member is then orthogonal to this pivot axis. The irrigation channel is substantially parallel to the plane when at least most portions of the irrigation channel are parallel to the plane or enclose an angle thereto that is not greater than 5 degrees or not greater than 10 degrees. Therefore, irrigation liquid flowing through the irrigation channel mainly has directions of flow that are parallel to the plane or at a small angle to the plane.

A manipulation device as described here moreover comprises in particular a locking spur, which is connected or coupled in a mechanically rigid manner to the second grip member, such that a movement of the second grip member relative to the first grip member is associated with a movement of the locking spur relative to the first grip member; a locking device, which is connected mechanically to the first grip member and is movable relative to the first grip member between a release position, without interaction with the locking spur, and a locking position, in which the locking device can be connected to the locking spur with form-fit or force-fit engagement; and a release device, which is manually movable between a release position and a locking position, and which is coupled mechanically to the locking device in such a way that the locking device adopts its release position when the release device adopts its release position, and that the locking device adopts its locking position when the release device adopts its locking position.

The locking spur is in particular a spur having a plurality of locking grooves or a plurality of locking teeth. The locking device comprises in particular a latching pawl, which can engage in one of the plurality of locking grooves or can engage behind one of the plurality of locking teeth. In the locking position of the locking device, the locking device can be connected to the locking spur with form-fit or force-fit engagement. It is possible to have a range of particularly wide-opened positions of the second grip member relative to the first grip member, in which range the locking spur does not reach as far as the locking device and the latching pawl does not engage in a locking groove or engage behind a locking tooth.

The locking device and the locking spur are designed in particular such that, in the locking position of the locking device, a movement of the grip members away from each other is suppressed by form-fit or force-fit engagement. By contrast, a substantial movement of the grip members towards each other can be facilitated. This can be facilitated, for example, by an asymmetric configuration of locking grooves and latching pawl.

The release device comprises in particular a release lever having a first arm which is directly manually actuatable, specifically movable, and a second arm which can be formed in one piece with the locking device or can be connected mechanically rigidly to the locking device or can be mechanically coupled to the locking device. The release lever is designed and arranged in particular such that it extends through the first grip member, the directly manually actuatable first arm of the release lever is arranged at a side of the first grip member facing away from the second grip member, and the arm of the release lever formed in one piece with or mechanically connected to or mechanically coupled to the locking device is arranged at a side of the first grip member facing towards the second grip member.

In a manipulation device as described here, a first portion of the irrigation channel in particular connects the irrigation port to the release device.

This means that an end of the first portion, a first outlet of the irrigation channel, is arranged on the release device. The first portion of the irrigation channel connects the irrigation port in particular to a region of the release device that is provided and designed for direct manual actuation.

The release device, in particular the region of the release device provided for direct manual actuation, is particularly exposed to contamination, on account of the medical personnel constantly touching it with their hands, which are often contaminated with blood, other bodily fluids or tissue residues. Delivery of irrigation liquid to the release device can facilitate cleaning of the release device.

In a manipulation device as described here, the first portion of the irrigation channel in particular connects the irrigation port to a recess which at least partially receives the release device in the release position of the latter.

The recess is designed in particular as a groove, the shape of which corresponds to the shape of the region of the release device that is directly manually actuatable. In the release position of the release device, the directly manually actuatable region of the release device largely or entirely fills the recess, such that only a narrow gap remains through which irrigation liquid can flow. An outlet of the irrigation channel is in particular arranged partially or completely in the recess, such that a greater or lesser amount of irrigation liquid can flow into the recess depending on the position of the release device.

In a manipulation device as described here, the release device is designed in particular as a lever which is pivotable about a pivot axis, with a directly manually movable first arm, and with a second arm which is connected mechanically rigidly to the first arm and is coupled mechanically to the locking device, wherein a first outlet of the irrigation channel is arranged closer to the pivot axis of the release device than to the free end of the first arm of the release device.

The arrangement of the first outlet of the irrigation channel closer to the pivot axis than to the free end of the first arm of the release device can facilitate a diversion of irrigation liquid to other regions of the manipulation device, in particular of the first grip member.

In a manipulation device as described here, the release device, depending on its position, diverts irrigation liquid, flowing through the first portion of the irrigation channel, to a greater or lesser extent into a second portion of the irrigation channel.

In a manipulation device as described here, the release device, depending on its position, diverts irrigation liquid, flowing through the first portion of the irrigation channel, to a greater or lesser extent into a second portion of the irrigation channel, a cross section of the second portion of the irrigation channel being partially delimited by the release device.

The diverting action of the release device is based particularly on the fact that the release device, depending on its position, closes an outlet of the first portion of the irrigation channel to a greater or lesser extent. The second portion of the irrigation channel is in particular fluidically connected to the first portion at the outlet or near the outlet, or the first portion transitions at the outlet into the second portion of the irrigation channel.

The second portion of the irrigation channel in particular encloses, with the first portion of the irrigation channel, an angle in the range of between 60 degrees or 80 degrees and 100 degrees or 120 degrees. Irrigation liquid flowing from the first portion into the second portion of the irrigation channel therefore changes its direction of movement by an angle of between 60 degrees or 80 degrees and 100 degrees or 120 degrees.

If the release device does not close or only partially closes the opening at the end of the first portion of the irrigation channel, irrigation liquid flowing through the first portion of the irrigation channel can in particular emerge from the manipulation device straight or substantially straight through the outlet or cross into the recess for the release device or for its directly actuatable arm. If the release device partially or completely closes the outlet, irrigation liquid flowing through the first portion of the irrigation channel is partially or completely diverted into the second portion of the irrigation channel.

The diverting action of the release device can facilitate a variation of the flow of the irrigation liquid by manual actuation of the release device. Irrigation liquid delivered through the irrigation port can thus be delivered alternately to different regions of the manipulation device.

Since the cross section of the second portion of the irrigation channel is partially delimited by the release device, the irrigation liquid can flow around the release device and clean it. The cross section of the second portion of the irrigation channel is in particular at least partially delimited by the second arm of the release device.

In a manipulation device as described here, the second portion of the irrigation channel leads irrigation liquid in particular to a connection component, which mechanically couples or connects the second arm of the release device to the locking device.

The connection component couples in particular an end of the second arm of the release device, facing away from the pivot axis of the release device, to the locking device. The connection component is in particular mechanically connected by a respective hinge to the second arm of the release device and to the locking device. During the intended use of the manipulation device, the connection component is in particular subjected exclusively to tensioning.

Since the second portion of the irrigation channel leads irrigation liquid to the connection component, the latter, if appropriate together with the hinges by which it is connected to the release device and to the locking device, can also be cleaned by irrigation liquid flowing around it.

In a manipulation device as described here, the irrigation port is in particular moreover fluidically connected to a spatial region for receiving the locking spur.

In particular, the first portion of the irrigation channel transitions into the spatial region for receiving the locking spur, such that irrigation liquid introduced into the first portion of the irrigation channel can pass partially into the spatial region for receiving the locking spur and can flow through said region. The spatial region for receiving the locking spur is arranged on that side of the first grip member facing towards the second grip member and is exposed there to contamination to a comparatively lesser extent. Nevertheless, as an opening, and on account of its function, it is especially susceptible to contamination. It is therefore advantageous for this spatial region to be irrigated, especially from the inside outwards.

In a manipulation device as described above, the spatial region for receiving the locking spur conveys in particular irrigation liquid from the irrigation port to the locking device.

The locking device is in particular arranged directly outside the spatial region for receiving the locking spur and is thus irrigated and cleaned by irrigation fluid emerging from the spatial region.

A manipulation device as described here moreover comprises in particular a deactivation device, which is manually movable between a deactivation position and a working position, and which is mechanically coupled to the locking device in such a way that the locking device adopts its release position when the deactivation device adopts its deactivation position, and the locking device is manually movable between its locking position and its release position when the deactivation device adopts its working position.

The deactivation device is in particular mechanically coupled indirectly to the locking device, for example via the release device. Alternatively, the deactivation device can be mechanically coupled directly to the locking device, for example by means of a spring or another elastic or rigid connection component.

A manipulation device as described here moreover comprises in particular a deactivation device, which is manually movable between a deactivation position and a working position, and which is mechanically coupled to the release device in such a way that the release device adopts its release position when the deactivation device adopts its deactivation position, and the release device is manually movable between its locking position and its release position when the deactivation device adopts its working position.

The mechanical coupling between the deactivation device and the release device is affected, for example, by means of a spring or another elastic or rigid connection component. In the deactivation position of the deactivation device, the spring holds the release device in its release position. In the working position of the deactivation device, the spring exerts no force or only a slight force on the release device, such that the latter is movable between its locking position and its release position.

A manipulation device as described here moreover comprises in particular a third portion of the irrigation channel, which portion fluidically connects the irrigation port or the first portion of the irrigation channel to the deactivation device.

A manipulation device as described here moreover comprises in particular a deactivation device, which is manually movable between a deactivation position and a working position, and which is mechanically coupled to the locking device in such a way that the locking device adopts its release position when the deactivation device adopts its deactivation position, and the locking device is manually movable between its locking position and its release position when the deactivation device adopts its working position, and a third portion of the irrigation channel for delivering irrigation fluid to the deactivation device.

The deactivation device is in particular mechanically coupled indirectly to the locking device, specifically via the release device. If the deactivation device is in its deactivation position, it holds the release device in the release position of the latter and thus also indirectly holds the locking device in its release position.

The third portion of the irrigation channel in particular connects the irrigation port to the deactivation device directly or indirectly via the first portion or via the first portion and the second portion. Through the delivery of irrigation fluid to the deactivation device, the deactivation device can also be cleaned by the irrigation fluid.

In a manipulation device as described here, the release device, depending on its position, in particular diverts irrigation liquid, flowing through the first portion of the irrigation channel, to a greater or lesser extent into the third portion of the irrigation channel.

The diverting action of the release device is based particularly on more or less complete closing or shifting of one or more alternative flow paths. In particular, the release device, depending on its position, closes an outlet through which irrigation liquid flowing through the first portion of the irrigation channel can emerge from the manipulation device, to a greater or lesser extent, such that the irrigation liquid flows to a greater or lesser extent into the third portion of the irrigation channel.

In a manipulation device as described here, the third portion of the irrigation channel in particular crosses the release device, wherein the cross section of the release device in the region of the third portion of the irrigation channel is narrower than the cross section of the release device in a region provided for direct manual actuation.

The third portion of the irrigation channel crosses the release device in particular at an angle of between 30 degrees and 90 degrees with respect to the main direction of extent of the release device at this location.

The width of the cross section of the release device, measured in a direction orthogonal to the main directions of extent of the arms of the release device and orthogonal to the third portion of the irrigation channel, is smaller in the region of the third portion of the irrigation channel than in a region of the release device provided for direct manual actuation. In particular, the width of the cross section of the release device in the region of the third portion is smaller, by at least a quarter or at least a third or at least half, than in a region provided for direct manual actuation.

By virtue of the cross section of the release device being smaller in the region of the third portion of the irrigation channel, irrigation liquid can flow past the release device, from one side of the release device to an opposite side of the release device, and thus in particular to the deactivation device.

In a manipulation device as described here, the third portion of the irrigation channel, in the region in which it crosses the release device, is in particular forked and leads past two mutually opposite sides of the release device.

The forked design of the third portion of the irrigation channel can facilitate complete irrigation of the release device at this location. Moreover, the forked design of the third portion of the irrigation channel can increase the overall cross section available to the irrigation liquid and thereby reduce the flow resistance. After it has passed the release device, irrigation fluid flowing in the third portion of the irrigation channel can once again fill a single, easily continuous cross section.

A manipulation device as described here moreover comprises in particular a main body, which forms a grip member and has a cavity, and a carrier structure, which is arranged in the cavity in the main body and is connected in a mechanically rigid manner to the main body, wherein shafts, which define pivot axes of at least either the locking device or the release device or the deactivation device, are secured or guided in the carrier structure.

A manipulation device as described here moreover comprises in particular a main body, which forms a grip member and has a cavity, and a carrier structure, which is arranged in the cavity in the main body and is connected in a mechanically rigid manner to the main body, wherein hinges, which define pivot axes of at least either the locking device or the release device or the deactivation device, are provided in the carrier structure.

A manipulation device as described here moreover comprises in particular a main body, which forms a grip member and has a cavity, and a carrier structure, which is arranged in the cavity in the main body and is connected in a mechanically rigid manner to the main body, wherein shafts, which define pivot axes of at least either the locking device or the release device or the deactivation device, are secured or guided in the carrier structure, wherein, in the region of the third portion of the irrigation channel, the carrier structure does not cover the surfaces of the cavity in the main body.

Since the carrier structure in the region of the third portion of the irrigation channel does not cover the surfaces of the cavity in the main body, the cross section of the third portion of the irrigation channel initially remains free inside the cavity in the main body.

In a manipulation device as described here, the main body has, at a first side, a first opening, in which the locking device is arranged and in which the locking spur can engage, said opening facing the cavity, wherein the main body has, at a second side facing away from the first side, a second opening, through which the release device engages, said second opening facing the cavity, and a third opening, through which the deactivation device engages, said third opening facing the cavity, wherein a web separates the second opening and the third opening, and wherein the web partially delimits the cross section of the third portion of the irrigation channel.

The web thus guides the irrigation fluid in the third portion, such that the irrigation fluid is conveyed completely or to a large extent to the deactivation device. Without the web, the second opening and the third opening would transition into each other, and irrigation fluid would be able to emerge from the manipulation device without having achieved its full effect.

A medical instrument comprises a manipulation device as described here.

The medical instrument moreover comprises in particular a shaft which can be connected to the manipulation device permanently and which cannot be released from it without destruction. Alternatively, the shaft can be connected such that it can be separated from the manipulation device without destruction, that is to say reversibly, and also in particular without use of tools, and can be connected to it again. The medical instrument can moreover have a tool at the distal end of the shaft, for example a tool for grasping, squeezing and/or cutting. The tool can be connected to the distal end of the shaft permanently and cannot be separated from it without destruction. Alternatively, the tool can be separated from the distal end of the shaft without destruction, that is to say reversibly, and also in particular without use of tools, and can be connected to it again.

### BRIEF DESCRIPTION OF THE DRAWINGS

Embodiments are explained in more detail below with reference to the accompanying figures, in which:

FIG. 1 shows a schematic view of a microinvasive medical instrument;

FIG. 2 shows a schematic enlarged view of a manipulation device of the instrument from FIG. 1;

FIG. 3 shows a schematic view of a section through the manipulation device from FIG. 2;

FIG. 4 shows a further schematic view of a section through the manipulation device from FIGS. 2 and 3;

FIG. 5 shows a further schematic view of a section through the manipulation device from FIGS. 2 to 4;

FIG. 6 shows a further schematic view of a section through the manipulation device from FIGS. 2 to 5;

FIG. 7 shows a further schematic view of a section through the manipulation device from FIGS. 2 to 6; and

FIG. 8 shows a further schematic view of a section through the manipulation device from FIGS. 2 to 7.

### DETAILED DESCRIPTION OF THE REPRESENTATIVE EMBODIMENTS

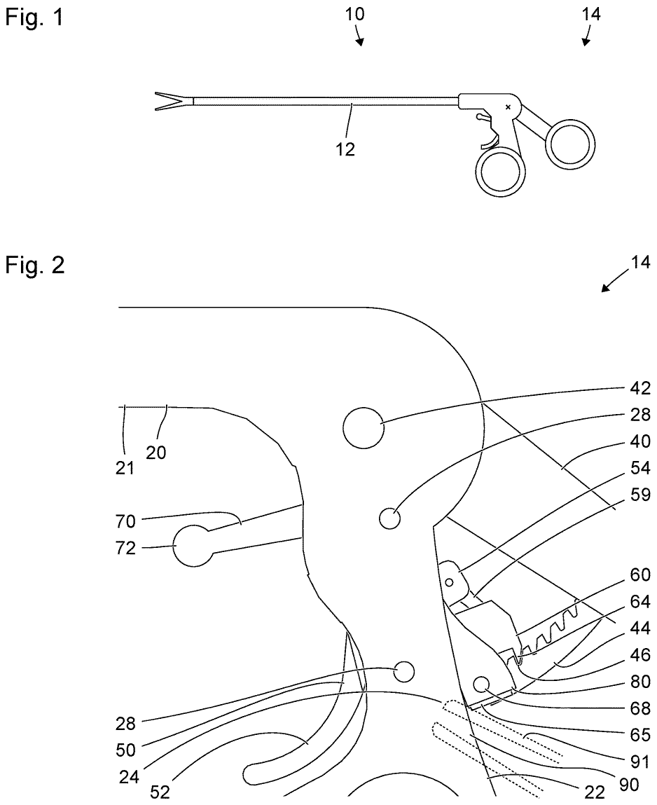

FIG. 1 shows a schematic view of a microinvasive medical instrument 10 having a long and thin shaft 12 and a tool at the distal end of the shaft 12, which can be inserted partially or completely into a natural or artificial cavity in a body of a human or animal patient. At the proximal end of the shaft 12, a manipulation device 14 is provided by means of which the medical instrument, especially the tool at the distal end of the shaft 12, can be controlled manually. The manipulation device 14 can be connected to the shaft 12 in such a way that the shaft 12 and the manipulation device 14 cannot be separated in a non-destructive and reversible manner. Alternatively, the medical instrument 10 is designed, for example, such that the shaft 12 and the manipulation device 14 can be separated from each other without use of tools and without destruction, that is to say reversibly, and can be connected to each other again. For this purpose, for example, a bayonet connection and/or a latching connection can be provided.

FIG. 2 shows a schematic and, in relation to FIG. 1, enlarged view of the manipulation device 14. The manipulation device 14 comprises a main body 20, which in particular is originally produced in one piece, for example by a casting method, a cutting method, 3D printing or another additive method. The main body can be made of surgical stainless steel or another metal, ceramic, plastic or one or more other materials.

The main body 20 has a distal region 21, which can be connected to the shaft 12 (compare FIG. 1) or can transition into the latter. The main body 20 moreover forms a grip member 22 which, for example similarly to scissors, can have an eye for accommodating one or more fingers of medical personnel. The grip member 22 is angled in relation to the distal end region 21 of the main body, in particular by an angle of approximately 45 degrees to 90 degrees, in particular 60 degrees to 90 degrees.

The manipulation device 14 moreover has a pivotable grip member 40, which is pivotable with respect to the main body 20 about a pivot axis which is defined by a hinge 42 and which is orthogonal to the drawing plane of FIG. 2. A locking spur 44 having a plurality or multiplicity of locking grooves 46 is connected rigidly to the pivotable grip member 40.

The manipulation device 14 moreover has a release lever 50. A first arm 52 of the release lever 50 is provided and designed for direct manual actuation. The first arm 52 of the release lever 50 is arranged at a side of the first grip member 22 facing away from the second grip member 40. The release lever 50 is arranged partially in a cavity 30 in the main body 20. A second arm 54 of the release lever 50 is connected rigidly to the first arm 52 of the latter and together with this is pivotable about a pivot axis that is orthogonal to the drawing plane of FIG. 2 and defined by a pivot hinge not visible in FIG. 2. The release lever 50 has a second arm 54, of which the end facing away from the first arm 52 is arranged at a side of the first grip member 22 facing towards the second grip member 40.

The release lever 50 is mechanically coupled to a locking device 60 by a connection component 59. One end of the connection component 59 is connected by a hinge to that end of the second arm 54 facing away from the first arm 52. The other end of the connection component 59 is connected to the locking device 60 by a further hinge. The connection component 59 is in particular substantially bar-shaped or rod-shaped.

The locking device 60 comprises a latching pawl 64 for engaging in any one of the locking grooves 46 of the locking spur 44. The locking device 60 is pivotable about a pivot axis which is defined by a hinge 68 and which is orthogonal to the drawing plane of FIG. 2. In the locking position of the locking device 60 as shown in FIG. 2, the latching pawl 64 of the locking device 60 engages in one of the locking grooves 46 of the locking spur 44 and thereby prevents, by form-fit engagement, an increase of the angle between the second grip member 40 and the first grip member 22. In a release position (not shown in FIG. 2) of the locking device, the latching pawl 64 does not engage in one of the locking grooves 46 of the locking spur 44, and the angle between the second grip member 40 and the first grip member 22 can be changed. As is described below, the locking device 60 can be moved to its release position (not shown in FIG. 2) by manual actuation of the first arm 52 of the release lever 50. Moreover, the latching pawl 64 of the locking device 60 and the locking grooves 46 of the locking spur 44 are designed such that a reduction of the angle between the second grip member 40 and the first grip member 22 is always possible, by means of the locking device 60 moving temporarily from its locking position in the direction of its release position.

The manipulation device 14 moreover has a deactivation lever 70, which is likewise partially arranged in the aforementioned cavity 30 in the main body 20 and is coupled mechanically there to the release lever 50. The deactivation lever 70 is pivotable about a pivot axis, which is defined by a hinge not visible in FIG. 2 and is orthogonal to the drawing plane of FIG. 2, between a deactivation position and a working position (or a plurality of working positions, in particular a range of working positions).

The deactivation lever 70 is coupled mechanically to the release lever 50 in such a way that the release lever 50 is held in its release position (not shown in FIG. 2) and, therefore, the locking device is directly also held in its release position (not shown in FIG. 2) when the deactivation lever adopts its deactivation position (not shown in FIG. 2). The deactivation lever 70 is coupled mechanically to the release lever 50 in such a way that the release lever 50 can be moved between its locking position (shown in FIG. 2) and its release position (not shown in FIG. 2), and therefore the locking device 60 can also be moved between its locking position (shown in FIG. 2) and its release position (not shown in FIG. 2), when the deactivation lever 70 adopts its working position or the position of the deactivation lever 70 lies within the range of working positions.

A carrier structure 80 is arranged in said cavity 30 (not visible in FIG. 2) in the main body 20 and protrudes out of the cavity in the region of the locking device 60. The locking device 60 is pivotable about a pivot axis which is defined by a hinge 68 between the locking device 60 and the carrier structure 80 and which is orthogonal to the drawing plane of FIG. 2.

The cavity (not visible in FIG. 2) and the carrier structure 80 each have in particular two mutually opposite plane and parallel surface regions, wherein the outer surface regions of the carrier structure 80 bear on the corresponding inner surface regions of the cavity. Locating pins 28, which extend orthogonally with respect to the drawing plane of FIG. 2 and whose ends are visible in FIG. 2, fix the carrier structure 80 in the cavity.

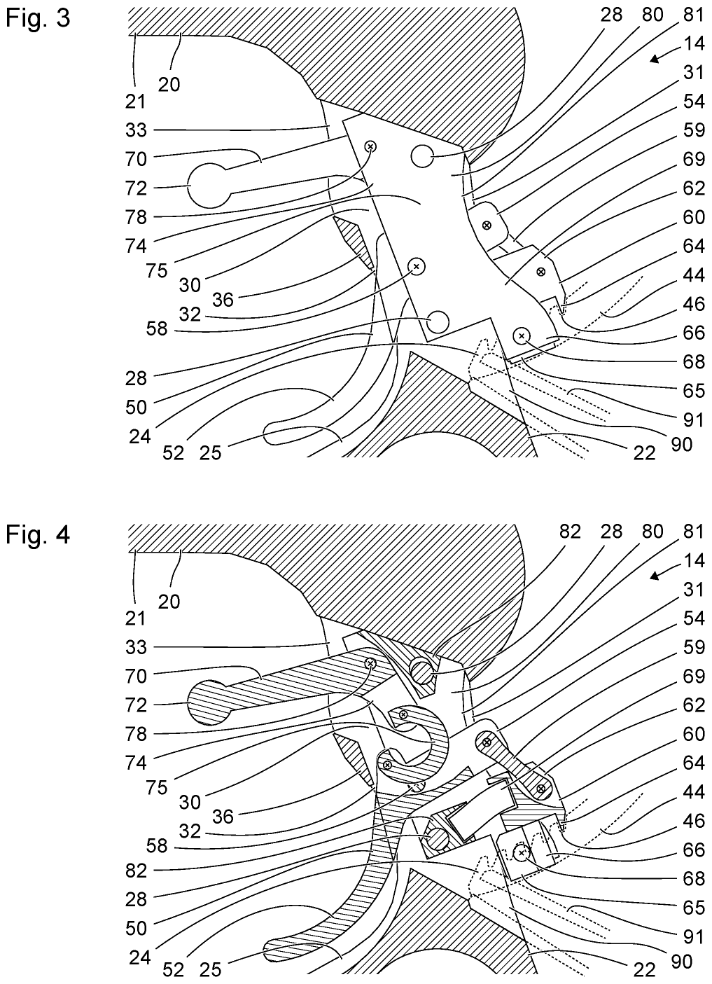

FIG. 3 shows a schematic view of a section through the main body 20 of the manipulation device 14 from FIGS. 1 and 2. The main body 20 is shown sectioned along a section plane parallel to the drawing plane of FIG. 2. The pivotable second grip member 40 (compare FIG. 2) is not shown in FIG. 3. Details of its hinged connection to the main body 20 and of its mechanical coupling to a pull rod and/or to other devices of the medical instrument, are also not shown in FIG. 3. The aforementioned cavity 30 in the main body 20 is visible in FIG. 3. The carrier structure 80 is arranged in the cavity 30 and partially fills the cavity 30. The carrier structure 80, the release lever 50, the locking device 60 and the deactivation lever 70 are not shown in section in FIG. 3, but in a plan view.

The main body 20 has three openings to the cavity 30. A first opening 31 to the cavity 30 is arranged at the side of the first grip member 22 facing towards the second grip member 40 (compare FIG. 2). The second arm 54 of the release lever 50 and the carrier structure 80 protrude through the first opening out of the cavity 30. The locking device 60 is arranged largely or entirely outside the cavity 30. A part of the cavity 30 that is not filled by the carrier structure 80 forms a spatial region 24 provided for receiving the locking spur 44. An edge region of the cavity 30 is moreover provided and designed as an irrigation port 90 for receiving a tube 91 (indicated by broken lines in FIG. 3) or a corresponding nozzle for delivery of irrigation fluid. The irrigation port is in particular designed as a concave acute-angled truncated cone according to the Luer standard.

Moreover, at its side facing away from the second grip member 40 (compare FIG. 2), the main body 20 has a second opening 32 and a third opening 33 towards the cavity 30. The second opening 32 and the third opening 33 are separated by a web 36. The release lever 50, specifically its first arm provided for direct manual actuation, protrudes from the cavity 30 through the second opening 32. The second opening 32 is adjoined by a groove 25 into the main body 20, which groove is provided and designed to partially receive the directly manually actuatable first arm 52 of the release lever 50. A change of the cross section of the first arm 52 of the release lever 50 in the region of the second opening 32 is indicated by a line. The first arm 52 of the release lever 50 has a greater width outside the cavity 30 than inside, as measured in a direction orthogonal to the drawing plane of FIG. 3.

A hinge 58 between the release lever 50 and the carrier structure 80 defines a pivot axis which is orthogonal to the drawing plane of FIG. 3. For reasons that are described with reference to FIG. 4 for example, the hinge 58 is formed by two short shafts or pins which connect on the one hand the side of the release lever facing away from the observer, and on the other hand the side of the release lever facing the observer, to the carrier structure 80.

The deactivation lever 70 protrudes from the cavity 30 through the third opening 33. A hinge 78 between the deactivation lever 70 and the carrier structure 80 defines a pivot axis which is orthogonal to the drawing plane of FIG. 3. The pivot hinge 78 is formed by a shaft or axle, of which an end is visible in FIG. 3.

FIG. 4 shows a schematic view of a section through the manipulation device 14 from FIGS. 1 to 3. The position of the section plane of FIG. 4 corresponds to that of the section plane of the main body 20 in FIG. 3. In contrast to FIG. 3, however, in FIG. 4 the release lever 50, the locking device 60, the deactivation lever 70 and the carrier structure 80 are also shown in section along the same plane.

The carrier structure 80 comprises two side parts or side-part regions 81, which are arranged parallel to the section plane of FIG. 4, and of which only one is visible in FIG. 4, specifically the one lying behind the section plane. Those surface regions of the side parts or side-part regions facing away from each other bear flat on the inner surface of the cavity. The carrier structure 80 moreover comprises two pillars 82 with irregular cross sections, which connect the side parts or side-part regions 81 to one another in a mechanically rigid manner. The locating pins 28, which fix the carrier structure 80 in the cavity 30, are arranged inside the pillars 82.

The second arm 54 of the release lever 50 is partially forked or is formed with a deep longitudinal groove parallel to the section plane of FIG. 4. An end of the connection component 59 and a part of a C-shaped spring 75 are arranged in this longitudinal groove. Since the pivot axis of the release lever 50, as defined by the pivot hinge 58, passes through this longitudinal groove, the pivot hinge 58 is formed by two short shafts on both sides of this longitudinal groove, that is to say respectively in front of and behind the section plane of FIG. 4.

The locking device 60 has a groove 65 which extends parallel to the section plane of FIG. 4 and which serves to receive the locking spur 44. The pivot hinge 68 is arranged in the region of this groove 65; the pivot axis defined by the pivot hinge 68 passes orthogonally through the groove 65. Therefore, as is mentioned above, the hinge 68 is formed by two short shafts or pins which, on both sides of this groove 65, that is to say respectively in front of and behind the section plane of FIG. 4, connect the locking device 60 to the carrier structure 80.

The locking device 60 moreover has a groove 62 parallel to the section plane of FIG. 4, in which groove 62 an end of the connection component 59 is arranged.

The ends of a spring 69 are arranged in a pocket in one of the pillars 82 of the carrier structure 80 or in a pocket in the locking device 60. The spring 69 is indicated in FIG. 4 only by its contours and is in fact designed in particular as a compressed helical spring.

FIG. 4 shows the deactivation lever 70 in a working position, the release lever 50 in its locking position, and the locking device 60 in its locking position.

An end 74 of the deactivation lever 70 facing away from the manually actuatable region 72 of the deactivation lever 70 is coupled to the release lever 50 by the C-shaped spring 75.

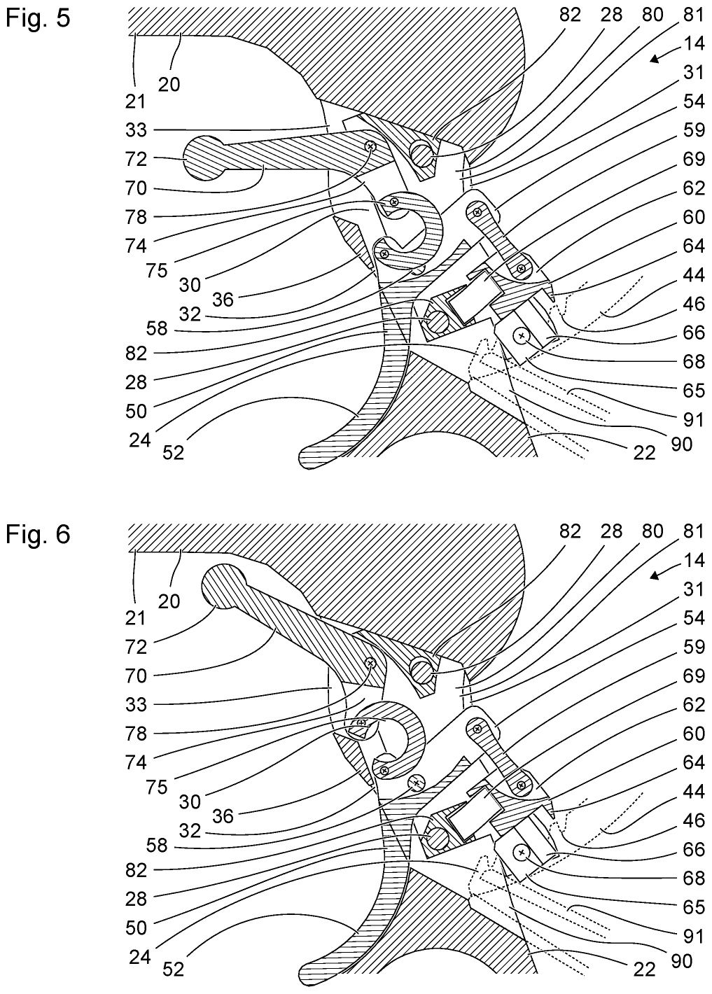

FIG. 5 shows a schematic view of a further section through the manipulation device 14 from FIGS. 1 to 4. The nature of the view, in particular the position of the section plane, corresponds to that of FIG. 4.

As in the situation or configuration shown in FIGS. 2 to 4, the deactivation lever 70 is also located in a working position in the situation or configuration shown in FIG. 5. The C-shaped spring 75 between the deactivation lever 70 and the release lever 50 does not enforce any particular position of the release lever. However, the situation or configuration shown in FIG. 5 differs from the configuration shown in FIGS. 2 to 4 in that the release lever 50 is moved towards the first grip member 22 by direct manual actuation, specifically by its first arm 52 being pressed, and adopts its release position in the groove 25 (compares FIGS. 3 and 4). On account of the mechanical coupling by means of the connection component 59, in this release position of the release lever 50 the locking device 60 also adopts its release position, in which the latching pawl 64 does not lock the locking spur 44.

In the release position of the locking device 60 as shown in FIG. 5, the spring 69 is compressed, counter to its restoring force, between the locking device 60 and the carrier structure 80. Therefore, the elastic restoring force of the spring 69 moves the locking device 60 and thus also the release lever 50 from their release positions shown in FIG. 5 back to their locking positions shown in FIG. 4, when the release lever 50 is not pressed manually, counter to the restoring force of the spring 69, to the release position shown in FIG. 5.

FIG. 6 shows a schematic view of a further section through the manipulation device 14 from FIGS. 2 to 5. The nature of the view, in particular the section plane, corresponds to that of FIGS. 4 and 5.

FIG. 6 shows the deactivation lever 70 in a deactivation position, in which the C-shaped spring 75 holds the release lever 50 in its release position and thus indirectly holds the locking device 60 in the release position of the latter. For this purpose, the C-shaped spring 75 is dimensioned between the deactivation lever 70 and the release lever 50 such that the torque exerted by it on the release lever 50 is greater than the inverse torque exerted on the release lever 50 by the spring 69 by way of the connection component 59. The C-shaped spring 75 moreover holds the deactivation lever 70 in its deactivation position shown in FIG. 6.

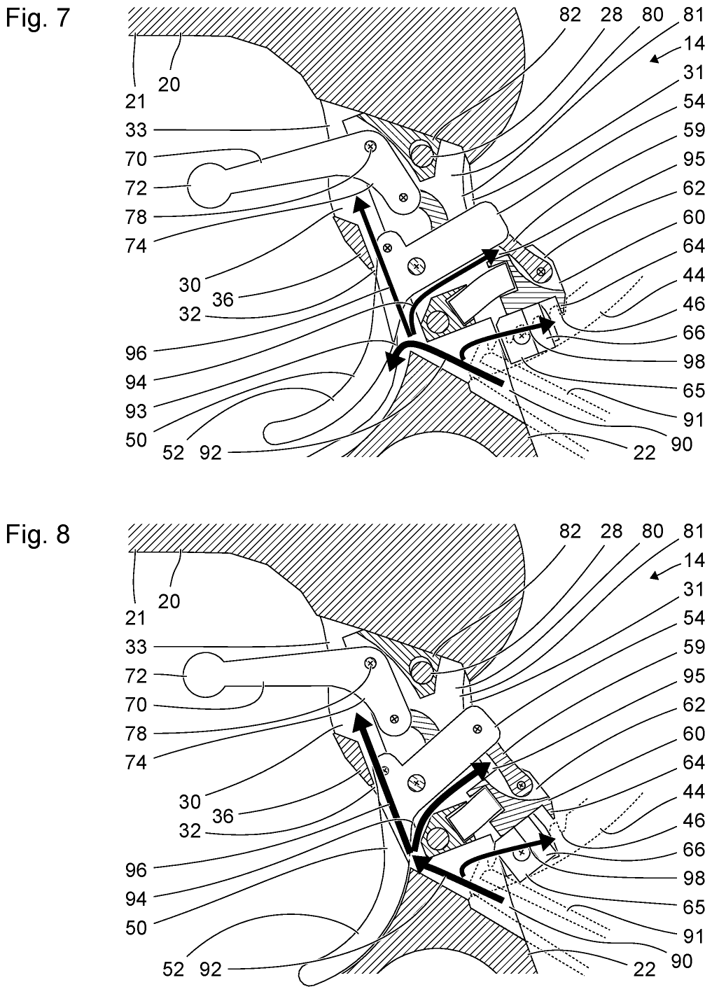

FIG. 7 shows a further view of a section through the manipulation device 14 from FIGS. 2 to 6. The nature of the view corresponds largely to that of FIGS. 4 to 6, although the release lever 50 and the deactivation lever 70 are not shown in section.

FIG. 7 shows the configuration already shown in FIG. 4, in which the deactivation lever 70 adopts a working position and the release lever 50 and the locking device 60 each adopt their respective locking positions. Several portions 92, 94, 96, 98 of a branched irrigation channel for irrigating and cleaning the manipulation device 14 by means of an irrigation fluid are indicated in FIG. 7. The portions 92, 94, 96, 98 of the irrigation channel are indicated by arrows, which indicate the direction of flow of the irrigation fluid.

A first portion 92 of the irrigation channel, starting from the irrigation port 90, leads substantially straight past the carrier structure 80 to an outlet 93, at which the irrigation fluid can emerge from the manipulation device 14 partially through the groove 25 and between the first grip member 22 and the first arm 52 of the release lever 50.

However, some of the irrigation fluid inside the manipulation device 14 continues through a second portion 94 of the irrigation channel between the second arm 54 of the release lever 50 and a pillar 82 of the carrier structure 80 to the connection component 59 and to a region of the locking device 60 facing away from the latching pawl 64, in order to emerge there from the manipulation device 14. The region between the edges of the side part or side-part region 81, the second arm 54 of the release lever, the locking device 60 and the connection component 59 thus forms the outlet 95 of the second portion 94 of the irrigation channel.

A further amount of the irrigation fluid flows along a third portion 96 past the release lever 50 on both sides (that is to say in front of and behind with respect to FIG. 7) to the deactivation lever 70. The third portion 96 of the irrigation channel is partially delimited by the web 36 and the opposite edges of the side parts or side-part regions 81 of the carrier structure 80. The third portion 96 of the irrigation channel leads to the third opening 33 of the cavity 30, which opening 33 functions mainly as an outlet for the irrigation fluid.

A fourth portion 98 of the irrigation channel branches off from the first portion 92 and leads through the spatial region provided for receiving the locking spur 44 (compare FIG. 2), and through the groove 65 in the locking device 60 for receiving the locking spur 44, to the latching pawl 64 and there leaves the manipulation device. The groove 65 for receiving the locking spur 44 in the locking device 60 is here at the same time the outlet of the fourth portion 98 of the irrigation channel.

FIG. 8 shows a further schematic view of a section through the manipulation device from FIGS. 2 to 7. The nature of the view corresponds to that of FIG. 7.

The configuration shown in FIG. 8 corresponds to the configuration shown in FIG. 5, in which the deactivation lever 70 adopts a working position and the release lever 50 and the locking device 60 adopt their release positions. In its release position shown in FIG. 8, the release lever 50 at least substantially closes the direct outlet 93 of the first portion 92 of the irrigation channel, such that considerably more irrigation liquid reaches the second portion 94, the third portion 96 and the fourth portion 98 of the irrigation channel. This is indicated by modified widths of the arrows representing the portions 94 and 96.

Although the invention and its advantages have been described in detail, it should be understood that various changes, substitutions and alterations can be made herein without departing from the scope of the invention as defined by the appended claims. The combinations of features described herein should not be interpreted to be limiting, and the features herein may be used in any working combination or sub-combination according to the invention. This de-scription should therefore be interpreted as providing written support, under U.S. patent law and any relevant foreign patent laws, for any working combination or some sub-combination of the features herein. Moreover, the scope of the present application is not intended to be limited to the particular embodiments of the process, machine, manufacture, composition of matter, means, methods and steps described in the specification. As one of ordinary skill in the art will readily appreciate from the disclosure of the invention, processes, machines, manufacture, compositions of matter, means, methods, or steps, presently existing or later to be developed that perform substantially the same function or achieve substantially the same result as the corresponding embodiments described herein may be utilized according to the invention. Accordingly, the appended claims are intended to include within their scope such processes, machines, manufacture, com-positions of matter, means, methods, or steps.

1. A manipulation device for a medical instrument, comprising:
a first grip member and a second grip member, that are movable relative to each other;
an irrigation port for receiving an irrigation liquid for cleaning the manipulation device between two uses; and
an irrigation channel that connects the irrigation port to one or more outlets, wherein the irrigation channel is arranged in the first grip member.

2. The manipulation device of claim 1, wherein the irrigation port is arranged at a side of the first grip member facing towards the second grip member.

3. The manipulation device of claim 2, wherein the irrigation port is arranged proximally with respect to a connection region of the grip members.

4. The manipulation device of claim 3, wherein the second grip member is movable relative to the first grip member in a plane, and wherein the irrigation channel runs substantially parallel to the plane.

5. The manipulation device of claim 4, further comprising:
a locking spur that is connected or coupled in a mechanically rigid manner to the second grip member, such that a movement of the second grip member relative to the first grip member is associated with a movement of the locking spur relative to the first grip member;
a locking device, that is connected mechanically to the first grip member and is movable relative to the first grip member between a release position, without interaction with the locking spur, and a locking position, in which the locking device can be connected to the locking spur with form-fit or force-fit engagement; and
a release device, that is manually movable between a release position and a locking position, and that is coupled mechanically to the locking device in such a way that the locking device adopts its release position when the release device adopts its release position, and that the locking device adopts its locking position when the release device adopts its locking position.

6. The manipulation device of claim 5, wherein a first portion of the irrigation channel connects the irrigation port to the release device.

7. The manipulation device of claim 6, wherein the first portion of the irrigation channel connects the irrigation port to a recess that at least partially receives the release device in the release position of the latter.

8. The manipulation device of claim 6, wherein the release device, depending on its position, diverts irrigation liquid, flowing through the first portion of the irrigation channel, to a greater or lesser extent into a second portion of the irrigation channel, and a cross section of the second portion of the irrigation channel is partially delimited by the release device.

9. The manipulation device of claim 7, wherein the release device, depending on its position, diverts irrigation liquid, flowing through the first portion of the irrigation channel, to a greater or lesser extent into a second portion of the irrigation channel, and a cross section of the second portion of the irrigation channel is partially delimited by the release device.

10. The manipulation device of claim 8, wherein the second portion of the irrigation channel leads irrigation liquid to a connection component, that mechanically couples or connects the second arm of the release device to the locking device.

11. The manipulation device of claim 9, wherein the second portion of the irrigation channel leads irrigation liquid to a connection component, that mechanically couples or connects the second arm of the release device to the locking device.

12. The manipulation device of claim 11, wherein the irrigation port is moreover fluidically connected to a spatial region for receiving the locking spur.

13. The manipulation device according to claim 12, further comprising:
a deactivation device, that is manually movable between a deactivation position and a working position, and that is coupled mechanically to the locking device in such a way that the locking device adopts its release position when the deactivation device adopts its deactivation position, and the locking device is manually movable between its locking position and its release position when the deactivation device adopts its working position; and
a third portion of the irrigation channel, for delivering irrigation fluid to the deactivation device.

14. The manipulation device of claim 13, wherein the release device, depending on its position, diverts irrigation liquid, flowing through the first portion of the irrigation channel, to a greater or lesser extent into the third portion of the irrigation channel.

15. The manipulation device of claim 14, wherein
the third portion (96) of the irrigation channel crosses the release device (50), and
the cross section of the release device in the region of the third portion of the irrigation channel is narrower than the cross section of the release device in a region provided for direct manual actuation.

16. The manipulation device of claim 15, wherein the third portion of the irrigation channel, in the region in which it crosses the release device, is forked and leads past two sides of the release device facing away from each other.

17. The manipulation device of claim 13, further comprising:
a main body, that forms a grip member and has a cavity;
a carrier structure, that is arranged in the cavity in the main body and is connected in a mechanically rigid manner to the main body,
wherein shafts, that define pivot axes of at least either the locking device or the release device or the deactivation device, are secured or guided in the carrier structure, wherein, in the region of the third portion of the irrigation channel, the carrier structure does not cover the surfaces of the cavity in the main body.

18. The manipulation device of claim 16, further comprising:
a main body, that forms a grip member and has a cavity;
a carrier structure, that is arranged in the cavity in the main body and is connected in a mechanically rigid manner to the main body,
wherein shafts, that define pivot axes of at least either the locking device or the release device or the deactivation device, are secured or guided in the carrier structure, wherein, in the region of the third portion of the irrigation channel, the carrier structure does not cover the surfaces of the cavity in the main body.

19. The manipulation device of claim 18, wherein
the main body has, at a first side, a first opening, in which the locking device is arranged, and in which the locking spur can engage, said opening facing the cavity;
the main body has, at a second side facing away from the first side, a second opening, through which the release device engages, said second opening facing the cavity, and a third opening, through which the deactivation device engages, said third opening facing the cavity; and
a web, that separates the second opening and the third opening, partially delimits the cross section of the third portion of the irrigation channel.
